(19) United States
(12) Patent Application Publication
(10) Pub. No.: US 2025/0136325 A1
(43) Pub. Date: May 1, 2025

(54) PACKAGING FOR CONTAINERS

(71) Applicants: West Pharmaceutical Services, Inc., Exton, PA (US); Daikyo Seiko Ltd., Tochigi (JP)

(72) Inventors: James J. THOMPSON, Sinking Spring, PA (US); Eleftherios HRISTOFAS, Lancaster, PA (US); Hideaki KAWAMURA, Tochigi (JP); Takayuki YOSHIDA, Tochigi (JP)

(21) Appl. No.: 18/913,266

(22) Filed: Oct. 11, 2024

Related U.S. Application Data
(60) Provisional application No. 63/546,522, filed on Oct. 30, 2023.

(51) Int. Cl. B65D 25/10

(52) U.S. Cl. CPC B65D 25/108

(57) ABSTRACT

A nest may have a horizontal base forming a plurality of openings and a plurality of tubular members extending downwardly from the horizontal base. Each of the tubular members may have a side wall defining a well in communication with an opening of the plurality of openings. In some embodiments, the side wall has a counterbore extending from the opening at an angle relative to a longitudinal axis of the tubular member, the counterbore having an inner surface defining a continuous circumference or perimeter. In some embodiments, the side wall has an inner surface with an upper portion defined by a series of radii of curvature providing a continuously curved surface extending from the opening and reducing a diameter of the well.

### CROSS REFERENCE TO RELATED APPLICATIONS

This application claims the benefit of U.S. Provisional Patent App. No. 63/546,522, filed Oct. 30, 2023, the disclosure of which is hereby incorporated by reference herein.

### TECHNICAL FIELD

The disclosure relates to packaging for retaining a plurality of containers. More specifically, the disclosure relates to nests configured to retain a plurality of containers for a medical or cosmetic substance and be nested in a tub.

### BACKGROUND

A nest is commonly used in the pharmaceutical fill/finish industry to hold a plurality of containers. The nest can be used with equipment during fill/finish and post-fill/finish operations. After being filled with a medical or cosmetic substance, the nest can be nested in a tub for packaging, shipping, and staging.

### SUMMARY

Many original equipment manufacturers (OEMs) produce pharmaceutical fill/finish equipment. The present inventors have recognized that it is beneficial for the OEMs' customers when suppliers of pharmaceutical containers (such as vials, ampoules, or cartridges) package those components in packaging that is industry standard, as it enables simplification across machine designs. Industry standard packaging may provide the ability to process the product and packaging with a simple format part change-over for existing machines, as opposed to requiring a new machine build. While the height-level requirements for container packaging are outlined, for example, in current ISO standards and respective industry standards, the detailed design of the packaging heavily influences its performance across machines from different OEMs.

Thus, an aspect of the present disclosure is directed to a nest having: a horizontal base forming a plurality of openings; and a plurality of tubular members extending downwardly from the horizontal base, wherein each of the tubular members have a side wall defining a well in communication with an opening of the plurality of openings, and the side wall has a counterbore extending from the opening at an angle relative to a longitudinal axis of the tubular member, the counterbore having an inner surface defining a continuous circumference or perimeter.

The nest may embody one or more of the following features. The angle of the counterbore may be about 10° to about 20° relative to the longitudinal axis of the tubular member. Each tubular member may have a substantially cylindrical portion extending downwardly from the counterbore at an angle of about 0.25° to about 2° relative to the longitudinal axis of the tubular member. The substantially cylindrical portion may have an inner surface defining a continuous circumference or perimeter. The horizontal base may have an upper surface that is flat. Each tubular member may include at least one bottom wall extending laterally from a bottom portion of the side wall. The at least one bottom wall may include a plurality of bottom walls formed by a circular central opening and a plurality of extension cutouts. The at least one bottom wall may include four bottom walls formed by four extension cutouts occurring at 90-degree intervals. Each of the extension cutouts may include a curved lateral side that approximates a curvature of the inner surface of the side wall. For each of the tubular members, an entirety of an inner surface of the side wall may be devoid of ribs between the opening and the at least one bottom wall. The side wall may extend completely around the wells and defines a circular profile or perimeter extending from the opening to the at least one bottom wall.

Another aspect of the present disclosure is directed to a system having: a plurality of containers enclosing a medical or cosmetic substance; the nest receiving the plurality of containers in the plurality of tubular members; and a tub receiving the nest.

Yet another aspect of the present disclosure is directed to a nest having: a horizontal base forming a plurality of openings; and a plurality of tubular members extending downwardly from the horizontal base, wherein each of the tubular members have a side wall defining a well in communication with an opening of the plurality of openings, and the side wall has an inner surface with an upper portion defined by a series of radii of curvature providing a continuously curved surface extending from the opening and reducing a diameter of the well.

The nest may embody one or more of the following features. The series of radii of curvature may include a first radius of curvature, a second radius of curvature, and a third radius of curvature. The first radius of curvature may define a convex first portion of an inner surface of the side wall, the second radius of curvature may define a concave second portion of the inner surface of the side wall, and the third radius of curvature may define a convex third portion of the inner surface of the side wall. The first radius of curvature may be less than the second radius of curvature and/or the third radius of curvature. The side wall may have an inner surface defining a continuous circumference or perimeter. Each tubular member may have a substantially cylindrical portion extending downwardly from the upper portion at an angle of about 0.25° to about 2° relative to a longitudinal axis of the tubular member. The substantially cylindrical portion may have an inner surface defining a continuous circumference or perimeter. The horizontal base may have an upper surface that is flat. Each tubular member may include at least one bottom wall extending laterally from a bottom portion of the side wall. The at least one bottom wall may include a plurality of bottom walls formed by a circular central opening and a plurality of extension cutouts. The at least one bottom wall may include four bottom walls formed by four extension cutouts occurring at 90-degree intervals. Each of the extension cutouts may include a curved lateral side that approximates a curvature of the inner surface. For each of the tubular members, an entirety of the inner surface of the side wall may be devoid of ribs between the opening and the at least one bottom wall. The side wall may extend completely around the wells and defines a circular profile or perimeter extending from the opening to the at least one bottom wall.

Yet another aspect of the present disclosure is directed to a system having: a plurality of containers enclosing a medical or cosmetic substance; the nest receiving the plurality of containers in the plurality of tubular members; and a tub receiving the nest.

### BRIEF DESCRIPTION OF THE DRAWINGS

Specific embodiments of the present disclosure are described below in the detailed description by way of example only and with reference to the accompanying drawings, in which:

FIG. 1A illustrates a perspective view of a nest according to the present disclosure.

FIG. 1B illustrates a side view of a long side of the nest of FIG. 1A.

FIG. 2 illustrates a perspective view of a tub configured to receive to receive the nest of FIG. 1.

FIGS. 3A-B illustrate a first embodiment of a well of the nest of FIG. 1.

FIGS. 4A-B illustrate a second embodiment of a well of the nest of FIG. 1.

FIGS. 5A-C illustrate a third embodiment of a well of the nest of FIG. 1.

FIGS. 6A-E illustrate an overview of embodiments of a bottom portion of the well of FIGS. 3A-5C.

The same reference numbers are used in the drawings and the following detailed description to refer to the same or similar parts.

### DETAILED DESCRIPTION

The present disclosure is directed to a ready-to-use product that packages containers in a nested format. The packaging is designed to interface with semi-automated or automated fill/finish equipment that manipulates the packaging in order to fill containers (such as flangeless containers, vials, ampoules, or cartridges) with a drug product, to apply a closure typically made from an elastomer, and to then seal various primary container components together in an aseptic manner.

The present disclosure is directed to improving the ability of the containers to be handled and processed on OEM fill/finish equipment. More specifically, the present disclosure is directed to minimizing or eliminating the need for the fill/finish equipment manufactures to develop custom format parts that enable the equipment to perform the desired unit operations with the constituent components of the primary container as part of the fill finish process. The packaging of the present disclosure enables the customer to switch between different sizes of containers with a minimum of different format parts or no format part change-over at all. Thus, the present disclosure enables a maximization of compatibility across different sizes of containers which enables easier customer adoption of the product. Further, the packaging enables a maximization of compatibility across a plurality of, for example, fill/finish machines and package handling methods offered by various OEMs throughout an industry, thereby enabling a broad range of customers to use the product and packaging.

A first feature of the present disclosure is directed to maximizing the amount of flat surface area on a top surface of the nest (planar to upper openings). The present inventors recognize that some prior art devices feature a reinforcing border that projects above the level of the horizontal base and/or extend around the perimeter of the nest. For example, reinforcing ribs often extend perpendicular to the perimeter border at selected locations. Further, each well may be positioned in prior art devices such that their respective tops extend a distance above the horizontal base. The net effect of this configuration is that there is limited surface area available on the horizontal base that could be utilized by the machine vendors to apply a means to temporarily attach a portion of the machine, such as a flexible or fixed automation arm that could lift the nest and containers out of the tub and relocate the components for a desired fill and finish unit operation. Thus, the present disclosure maximizes the amount of flat surface area on the top of the nest planar to the openings.

A second feature is directed to providing improved vial support while the vial is in a raised position with respect to the nest, where a bottom of the container is raised to an upper portion of the well. The present inventors recognize that prior art devices have geometry of integral ribs that are positioned at specific orientations within each of the wells that hold the respective vials. The length and position of the ribs create a situation that when the vials are lifted up a suitable distance from the nest, the vials do not have sufficient axial support and they could fall over when contacted by a part of the machine's format parts. The present disclosure provides embodiments of a well with an improved vial support while the vial is in a raised position with respect to the nest.

A third feature is directed to the shape of openings located at the bottom of the wells of the nest that afford appropriately configured equipment elements to move axially from below the nest upward and engage the bottom of the vials for the purpose of elevating the vials to a desired location.

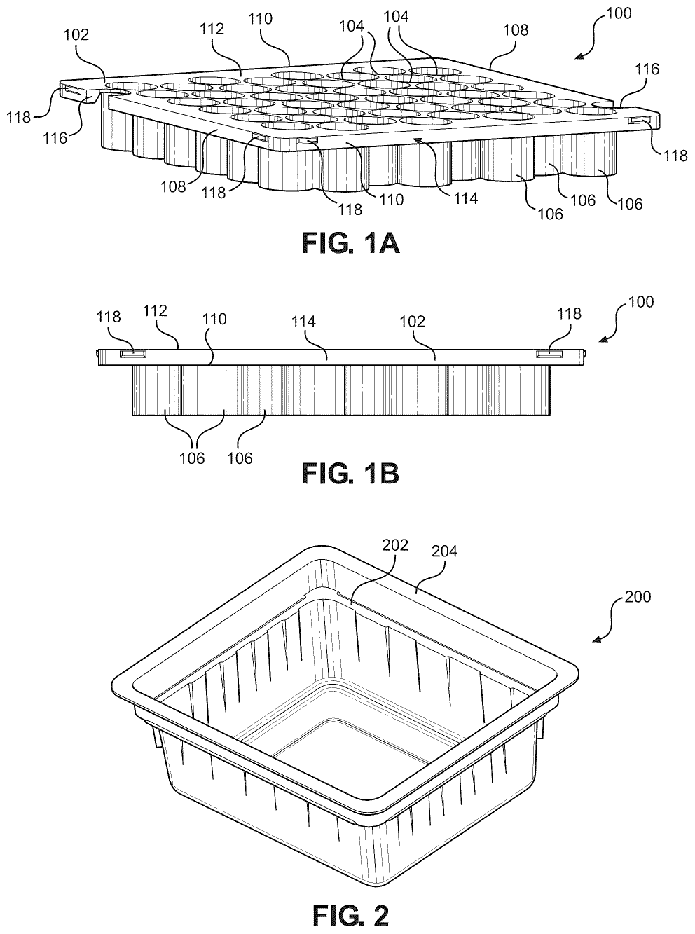

FIGS. 1A-2 illustrate an embodiment of a packaging system including a nest 100 and a tub 200, where the nest 100 may be received in the tub 200 in a nested arrangement. The packaging system may further include a plurality of containers (not shown) received in the nest 100. The containers may be vials, ampoules, or cartridges and may enclose a medical or cosmetic substance. The containers may have a generally cylindrical shape and may be made of a plastic material or glass. The containers may be received in the nest 100 such that the containers do not extend vertically above a top surface of the tub 200.

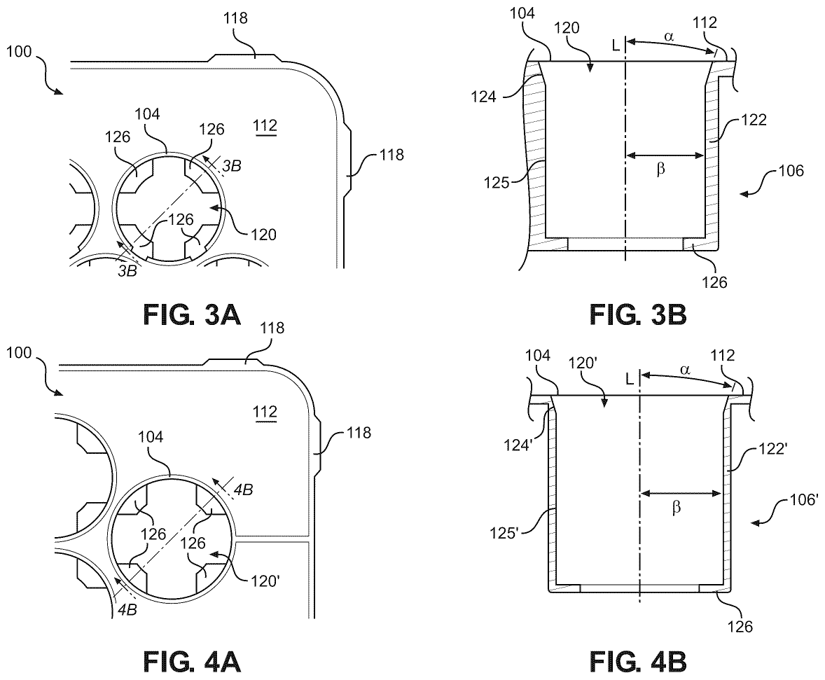

As illustrated, the nest 100 may have a horizontal base 102 defining a plurality of openings 104. A plurality of tubular members 106 may extend downwardly from the horizontal base 102 and define a plurality of wells (as illustrated in FIGS. 3A-5C) in communication with respective openings 104. The horizontal base 102 may be supported by an inner shoulder 202 of the tub 200 when the nest 100 is nested in the tub 200. The tub 200 may be sealed by a film or sheet (not shown) after insertion of the nest 100. A peripheral edge of the sheet may be attached to a rim 204 of the tub 200 with an adhesive to seal the interior of the tub 200.

The horizontal base 102 may have a pair of short side edges 108 and a pair of long side edges 110, defining a substantially rectangular profile. The nest 100 may define any number of wells for retaining a plurality of containers, each well for receiving and/or retaining one of the containers. For example, in some embodiments, the nest 100 may define forty-eight wells arranged in eights rows of wells, with the rows parallel to each other along the long side edges 110, and each row having six wells aligned along the short side edges 108. In some embodiments, the nest 100 may define one hundred wells arranged in ten rows of wells, with the rows parallel to each other along the long side edges 110, and each row having ten wells aligned along the short side edges 108. Other variations of the arrangement of rows of wells may include, for example, six by eight, eight by six, four by six, five by five, four by four; all dependent on the associated containers to be retained as well as requirements of particular OEMs. The rows of wells in each of the embodiments may overlap to optimize the surface area of the horizontal base 102 and provide a compact shape. The arrangement of the wells may allow containers to be arranged in a close-packed arrangement, and the pattern itself enables a nest of closures (not shown) arranged in the same pattern to be positioned above the nest 100, and automatic closing/stoppering of the containers to be performed in a single operation of the machine. Further, the close-packed arrangement of the wells presents the containers is a fixed orientation permitting equipment manufactures to individually or collectively select and handle the containers for various fill/finish unit operations.

The horizontal base 102 may have an upper surface 112 extending around the plurality of openings 104. As illustrated in FIGS. 1A-B, the upper surface 112 may be flat and spacious to facilitate handling by equipment. The upper surface 112 may be flat for its entire surface area between the side edges 108, 110 and be devoid of any ridges or protrusions extending vertically from the upper surface 112. In an illustrative embodiment, the flat surface area of the upper surface 112 may provide an improved landing for suction cups to enable the automatic lifting of the nest 100 out of the tub 200.

The horizontal base 102 may have a vertical wall 114 extending along a perimeter of the nest 100 and forming the side edges 108, 110 on the underside of the upper surface 112. The vertical wall 114 may provide rigidity to the nest 100 and prevent the horizontal base 102 from bending, warping, or flexing. Ribs (not shown) may be provided on the underside of the horizontal base 102 and extend parallel and/or perpendicularly of the vertical wall 114. The ribs may provide additional rigidity to the nest 100 and/or enable the nest 100 to be better secured onto machine components when the machines are employing features such as pins for locating, guiding, and providing interference of movement. The ribs may connect the tubular members 106.

The horizontal base 102 may further include a plurality of cutouts 116 configured to enable manual lifting of the nest 100 out of the tub 200 by an operator using fingers and/or centering of the nest 100 onto subsequent machine components when being manipulated either manually or automatically. For example, the horizontal base 102 may include a pair of the cutouts 116, with each being through the upper surface 112 and the vertical wall 114 along one of the short side edges 108 to facilitate handing and/or alignment.

The nest 100 may further include a plurality of tabs 118 extending laterally from the vertical wall 114. The plurality of tabs 118 may enable modification of the injection mold tooling resulting in changes to the overall length and width of the nest 100 without requiring the fabrication of new tooling and without affecting the thickness of vertical wall 114. For example, the tabs 118 may extend laterally from the horizontal base 102, perpendicularly from the side edges 108, 110. The nest 100 may include two tabs 118 extending from each of the side edges 108, 110, for a total of eight tabs 118. Each of the tabs 118 may be located near the corners formed by the side edges 108, 110.

The nest 100 may be made from plastic, specifically a plastic material demonstrated to resist abrasion while in contact with the containers, as well as to not cause scratching of the containers when made of glass or plastic, during the various conditions experienced by the nest 100 and the vials during the shelf life of the product, including sterilization, storage, shipping, container insertion, and container removal. For example, the nest 100 may be made from injection molding of plastic resin raw material. In other embodiments, the plastic may be, for example, polypropylene (PP), high density polyethylene (HDPE), polycarbonate (PC), polystyrene (PS), Polyacetal (POM). However, the nest 100 may be made from non-plastic and/or other manufacturing techniques, such as 3D-printing, CNC machining, and casting, with additional subsequent steps, such as polishing or passivation, to achieve the desired finishes and surface properties.

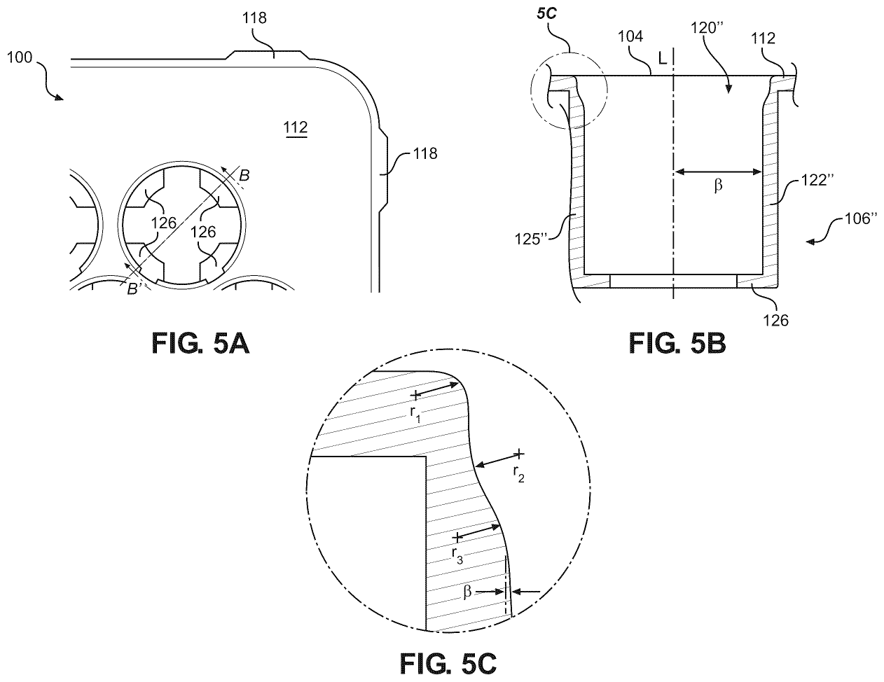

FIGS. 3A-B illustrate a first embodiment of the tubular members 106 defining a well 120, FIGS. 4A-B illustrate a second embodiment of the tubular members 106′ defining a well 120′, and FIGS. 5A-C illustrates a third embodiment of the tubular members 106″ defining a well 120″. Each of the tubular members 106, 106′, 106″ may have a side wall 122, 122′, 122″ may have an upper portion forming the opening 104 and extend downwardly from the horizontal base 102 to at least one bottom wall 126. The at least one bottom wall 126 may extend substantially laterally from a bottom portion of the side wall 122, 122′, 122″. The side wall 122, 122′, 122″ may be configured to guide one of the containers during insertion into the nest 100, and the at least one bottom wall 126 may be configured to support the container when inserted. Each of the side walls 122, 122′, 122″ may extend completely around the respective well 120, 120′, 120″ and define a circular profile or perimeter extending from the opening 104 to the at least one bottom wall 126. As further discussed herein, the circular profile or perimeter may have a variable diameter or circumference.

As illustrated in FIGS. 3A-B and 4A-B, the side wall 122, 122′ may have an inner surface defined by a series of straight lines of varying angles that collectively serve the purpose of centering the container upon ingress or egress, and impairing the ability of the vials to move radially within the well. The cross-section as illustrated in FIGS. 3B and 4B essentially eliminates an initial vertical cylinder positioned at the intersection of the upper surface 112 and the side wall 122, 122′ and replaces it with a series of angled sections that serve as a lead-in frustum followed by a slightly tapered cylinder to impair the axial movement of the vials within the receptacle.

For example, the side wall 122, 122′ may include a counterbore 124, 124′ extending from the opening 104 through the upper surface 112 and extending a distance into the well 120, 120′. The counterbore 124, 124′ may have an inner surface that extends at an angle α relative a longitudinal axis L of the tubular members 106, 106′. The angle α may be an acute angle. In some embodiments, the angle α may be about 10° to about 20°, for example about 15°. The counterbore 124, 124′ may provide a centering function to allow the container to be placed into the well 120, 120′ even when a vertical center axis of the container is slightly not aligned with the longitudinal axis L. Furthermore, previous designs feature a plurality of ribs positioned at selected locations around the circumference of each well. However, some fill/finish equipment designs raise the containers with respect to the nest 100 in order to facilitate machine unit operations. When this occurs the relative position of the bottom of the container and the upper edge of the guide ribs are very close to each other. The relative axial stability of the container is impaired and there is increased risk of the container in the prior art nest not remaining in a suitably vertical orientation to facilitate fill/finish unit operations. Thus, the inner surface of the counterbore 124, 124′ may have a continuous circumference or perimeter without interruptions to serve as the receptacle for the vials, by omitting ribs (longitudinal and circumferential). For example, the circumference of the counterbore 124, 124′ may be circular having a generally decreasing inner diameter (e.g., frustoconical shaped) extending downwardly around the longitudinal axis L. The inner surface of the side wall 122, 122′ may have a substantially cylindrical portion 125, 125′ extending downwardly to the at least one bottom wall 126. The substantially cylindrical portion 125, 125′ may extend at an angle β relative the longitudinal axis L of the tubular members 106, 106′. The angle β may be about 0.25° to about 2°, for example about 0.5°. The angle β may guide insertion of the container into the tubular members 106, 106′. In some embodiments, the substantially cylindrical portion 125, 125′ may be sized to provide sufficiently space from an outer surface of the container such that the container may be raised and lowered without any retention forces from the substantially cylindrical portion 125, 125′ in order to prevent scratching of the container. However, in other embodiments, the angle β may provide a frictional engagement of at least the bottom of the container to prevent or reduce lateral movement. The substantially cylindrical portion 125, 125′ may similarly have a continuous circumference or perimeter without interruptions to serve as the receptacle for the containers, by omitting ribs (longitudinal and circumferential). For example, the circumference of the substantially cylindrical portion 125, 125′ may be circular having a generally decreasing inner diameter extending downwardly around the longitudinal axis L. Thus, for each of the tubular members 106, 106′, the entire inner surface of the side wall 122, 122′ may be devoid of ribs (longitudinal and circumferential) between the opening 104 and the at least one bottom wall 126.

As illustrated in the third embodiment of FIGS. 5A-C, the side wall 122″ of the tubular members 106″ may have an inner surface with an upper portion defined by a series of radii to provide the centering function. As illustrated in FIG. 5C, the inner surface of the side wall 122″ may have a convex first portion extending from the opening 104 and defined by a first radius of curvature r1. The inner surface of the side wall 122″ may have a concave second portion continuously extending from the first portion and defined by a second radius of curvature r2. The inner surface of the side wall 122″ may have a convex third portion continuously extending from the second portion and defined by a third radius of curvature r3. The first radius of curvature r1 may be less than the second radius of curvature r2 and/or the third radius of curvature r3. The second radius of curvature r2 may be less than the third radius of curvature r3. The series of radii of curvature may define a continuously curved surface extending from the opening 104 and reducing the diameter of the well 120′″ to a desired diameter. As further discussed above with reference to FIGS. 5A-5B, the continuous surface may have a continuous circumference or perimeter without interruptions to serve as the receptacle for the container, provide stability of the container, and centering of the container; omitting ribs (longitudinal and circumferential). For example, the circumference of the continuous surface defined by the series of radii may be circular having a generally decreasing diameter extending downwardly into the well 120″ around the longitudinal axis L of the tubular member 106″. The inner surface of the side wall 122″ of the well 120″ may have a substantially cylindrical portion 125″ extending continuously from the third portion tangentially at angle β with respect to the longitudinal axis L to the at least one bottom wall 126, as discussed with regard to the side wall 122, 122′ and incorporated herein for sake of brevity. As discussed above, the angle β may be about 0.25° to about 2°, for example about 0.5°. The substantially cylindrical portion 125″ may similarly have a continuous circumference or perimeter without interruptions to serve as the receptacle for the containers, by omitting ribs (longitudinal and circumferential). For example, the circumference of the substantially cylindrical portion 125″ may be circular having a generally decreasing inner diameter extending downwardly around the longitudinal axis L of the well 120″. Thus, for each of the tubular members 106″, the entire inner surface of the side wall 122″ may be devoid of ribs (longitudinal and circumferential) between the opening 104 and the at least one bottom wall 126.

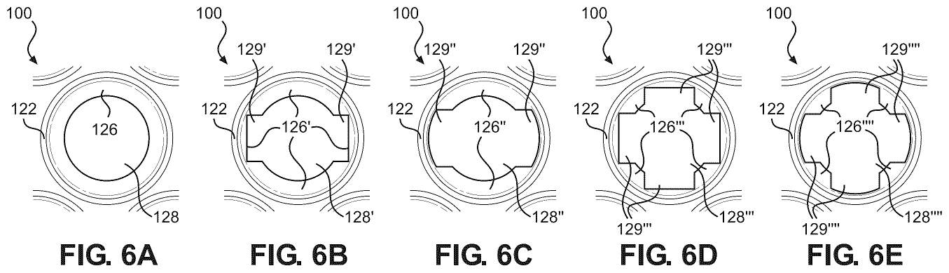

FIGS. 6A-E illustrate a plurality of embodiments of the at least bottom wall 126. The at least bottom wall 126 may form a surface for the container to sit on when inserted into the well 120. Each of the embodiments of the at least bottom wall 126 may form an opening 128 at the bottom of the well 120 to allow machine components to contact the bottom of the container to change its height position relative to the nest 100.

As illustrated in FIG. 6A, a first embodiment of the at least one bottom wall 126 may be a continuous annular wall defining a circular central opening 128.

As illustrated in FIG. 6B, a second embodiment of the at least one bottom wall 126′ may have a circular central opening 128′ and a pair of extension cutouts 129′ occurring at 180-degree intervals. Each of the extension cutouts 129′ may have three straight sides such that a bottom wall 126′ may be formed between each of the sides of the extension cutouts 129′ and the side wall 122 of the well 120. Bottom walls 126′ that extend further inward may have a concave inner surface that defines the circular central opening.

As illustrated in FIG. 6C, a third embodiment of the at least one bottom wall 126″ may be similar to the second embodiment having a circular central opening 128″ and a pair of extension cutouts 129″ occurring at 180-degree intervals. However, each of the extension cutouts 129″ of the third embodiment may have a curved or rounded lateral side. For example, the curved or rounded lateral side may approximate or extend parallel to the curvature of the side wall 122. Thus, the third embodiment of the at least one bottom wall 126″ may have only a pair of walls on opposing sides of the central opening 128″ and extension cutouts 129″, with minimal or no bottom walls formed between the curved lateral sides of the extension cutouts 129″ and the side wall 122. The bottom walls 126″ may have a concave inner surface that defines the circular central opening 128″.

As illustrated in FIG. 6D, a fourth embodiment of the at least one bottom wall 126′″ may have a circular central opening and four extension cutouts 129′″ occurring at 90-degree intervals, creating a cross-shaped opening 128′″. The fourth embodiment may allow the use of a rod with a circular cross-section to lift the container at least partially out of the well or the use of a rod with crosswise extensions to lift the container at least partially out of the well 120 with added stability for the container, as the crosswise extensions would apply lifting force closer to the perimeter of the container as opposed to the center of the container. The fourth embodiment may also form four bottom walls 126′″ in the form of tabs at the bottom of each well onto which container may rest. Bottom walls 126′″ that extend further inward may have a concave inner surface that defines the circular central opening 128′″.

As illustrated in FIG. 6E, a fifth embodiment of the at least one bottom wall 126″″ may be similar to the fourth embodiment having a circular central opening 128″″ and four extension cutouts 129″″ occurring at 90-degree intervals. Similar to the fourth embodiment, the fifth embodiment may allow the use of a rod with a circular cross-section to lift the container at least partially out of the well or a rod with crosswise extensions to lift the container at least partially out of the well with added stability for the container, as the crosswise extensions would apply lifting force closer to the perimeter of the vial as opposed to the center of the vial. The fifth embodiment may form four bottom walls 126″″ in the form of tabs at the bottom of each well onto which container may rest. However, each of the extensions of the fifth embodiment may have a curved or rounded lateral side. For example, the curved or rounded lateral side may approximate or extend parallel to the curvature of the side wall 122. Thus, the fifth embodiment of the at least one bottom wall 126″″ may have only four walls disposed radially around the central opening 128″″, with minimal or no bottom walls formed between the curved sides of the extensions and the side wall 122. The bottom walls 126″″ may have a concave inner surface that defines the circular central opening 128″″.

The rounding of the central openings 128, 128′, 128″, 128′″, and 128″″ and/or cutouts 129″, 129″″ may provide additional area available for fitting a lifting rod, even though the additional area gained is small. A wider lifting rod allows the lifting rod to contact the container further from its center, thereby increasing the stability of the container on the lifting rod and preventing the container from tilting of falling over once it has been lifted. Furthermore, the at least one bottom wall 126, 126′, 126″, 126′″ 126″″ needs to support the container throughout the entire life of the container within the well, including the empty container, as well as a filled, closed, and/or sealed container. Thus, the at least one bottom wall 126, 126′, 126″, 126′″ 126″″ must be able to hold the weight of the container with the medical substance, the weight of the closure, and the weight of the seal. The at least one bottom wall 126, 126′, 126″, 126′″126″″ may further need to counteract some additional temporary downward forces, for example applied during the container closing process, and which will vary in magnitude and duration depending on the container-closure combination and the machine speed.

The various embodiments of the tubular members 106, 106′, 106″ may implement any of the embodiments of the at least one bottom wall 126, 126′, 126″, 126′″, 126″″. However, the fifth embodiment of the at least one bottom wall 126″″ (as illustrated in FIG. 6E) may provide the most optimal area for the lifting rod and support for the container, and is further illustrated in FIGS. 3A, 4A, 5A.

It will also be appreciated by those skilled in the art that modifications can be made to the example embodiments described herein without departing from the invention. Structural features of systems and apparatuses described herein can be replaced with functionally equivalent parts or omitted entirely. Moreover, it will be appreciated that features from the embodiments can be combined with each other without departing from the disclosure.

What is claimed is:

1. A nest comprising:
a horizontal base forming a plurality of openings; and
a plurality of tubular members extending downwardly from the horizontal base,
wherein each of the tubular members have a side wall defining a well in communication with an opening of the plurality of openings, and the side wall has a counterbore extending from the opening at an angle relative to a longitudinal axis of the tubular member, the counterbore having an inner surface defining a continuous circumference or perimeter.

2. The nest of claim 1, wherein the angle of the counterbore is about 10° to about 20° relative to the longitudinal axis of the tubular member.

3. The nest of claim 1, wherein each tubular member has a substantially cylindrical portion extending downwardly from the counterbore at an angle of about 0.25° to about 2° relative to the longitudinal axis of the tubular member.

4. The nest of claim 1, wherein each tubular member includes at least one bottom wall extending laterally from a bottom portion of the side wall.

5. The nest of claim 4, wherein the at least one bottom wall includes a plurality of bottom walls formed by a circular central opening and a plurality of extension cutouts.

6. The nest of claim 5, wherein each of the extension cutouts includes a curved lateral side that approximates a curvature of the inner surface of the side wall.

7. The nest of claim 4, wherein, for each of the tubular members, an entirety of an inner surface of the side wall is devoid of ribs between the opening and the at least one bottom wall.

8. The nest of claim 4, wherein the side wall extends completely around the wells and defines a circular profile or perimeter extending from the opening to the at least one bottom wall.

9. A system comprising:
a plurality of containers enclosing a medical or cosmetic substance;
the nest of claim 1 receiving the plurality of containers in the plurality of tubular members; and
a tub receiving the nest.

10. A nest comprising:
a horizontal base forming a plurality of openings; and
a plurality of tubular members extending downwardly from the horizontal base,
wherein each of the tubular members have a side wall defining a well in communication with an opening of the plurality of openings, and the side wall has an inner surface with an upper portion defined by a series of radii of curvature providing a continuously curved surface extending from the opening and reducing a diameter of the well.

11. The nest of claim 10, wherein the series of radii of curvature includes a first radius of curvature, a second radius of curvature, and a third radius of curvature.

12. The nest of claim 11, wherein the first radius of curvature defines a convex first portion of an inner surface of the side wall, the second radius of curvature defines a concave second portion of the inner surface of the side wall, and the third radius of curvature defines a convex third portion of the inner surface of the side wall.

13. The nest of claim 10, wherein each tubular member has a substantially cylindrical portion extending downwardly from the upper portion at an angle of about 0.25° to about 2° relative to a longitudinal axis of the tubular member.

14. The nest of claim 13, wherein the substantially cylindrical portion has an inner surface defining a continuous circumference or perimeter.

15. The nest of claim 10, wherein each tubular member includes at least one bottom wall extending laterally from a bottom portion of the side wall.

16. The nest of claim 15, wherein the at least one bottom wall includes a plurality of bottom walls formed by a circular central opening and a plurality of extension cutouts.

17. The nest of claim 16, wherein each of the extension cutouts includes a curved lateral side that approximates a curvature of the inner surface.

18. The nest of claim 15, wherein, for each of the tubular members, an entirety of the inner surface of the side wall is devoid of ribs between the opening and the at least one bottom wall.

19. The nest of claim 15, wherein the side wall extends completely around the wells and defines a circular profile or perimeter extending from the opening to the at least one bottom wall.

20. A system comprising:
a plurality of containers enclosing a medical or cosmetic substance;
the nest of claim 10 receiving the plurality of containers in the plurality of tubular members; and
a tub receiving the nest.
